(19) United States
(12) Patent Application Publication
(10) Pub. No.: US 2025/0213535 A1
(43) Pub. Date: Jul. 3, 2025

(54) A PREMIXED ROOM TEMPERATURE STABLE COMPOSITION OF FAMOTIDINE FOR INTRAVENOUS BOLUS INJECTION AND METHOD THEREOF

(71) Applicant: MAIVA PHARMA PRIVATE LIMITED, Hosur (IN)

(72) Inventors: Alagumurugan Alagarswamy, Hosur (IN); Bhaskar Krishna Arumugam, Bangalore North (IN); Ramanathan Saiganesh, Chennai (IN); Senthilkumar Natarajan, Hosur (IN); Balamurugan S, Hosur (IN); Swetha Ramasamy, Devakottai (IN); Dhandapani K, Hosur (IN)

(21) Appl. No.: 18/846,161

(22) PCT Filed: May 19, 2023
(86) PCT No.: PCT/IB2023/055159

§ 371 Date: Sep. 11, 2024

(30) Foreign Application Priority Data
Sep. 21, 2022 (IN) 202241054109

(51) Int. Cl. A61K 31/426; A61K 9/00; A61K 47/02; A61K 47/18; A61K 47/20; A61K 47/26

(52) U.S. Cl. CPC A61K 31/426; A61K 9/0019; A61K 47/02; A61K 47/183; A61K 47/20; A61K 47/26

(57) ABSTRACT

A premixed room temperature stable composition 10 of famotidine 30 for intravenous bolus injection 20 and method thereof is provided. The composition includes 0.20 to 0.50 percentage by weight famotidine 30. The composition also includes a buffering agent 40. The composition further includes one or more tonicity adjusting agents 50. The composition includes potential of hydrogen value between 5.7 and 6.4. The composition is a premixed, isotonic, and ready to use solution. The composition is capable of being stored at room temperature.

### EARLIEST PRIORITY DATE

This Application claims priority from a complete patent application filed in India having patent application Ser. No. 20/224,1054109, filed on Sep. 21, 2022, and titled “A PREMIXED ROOM TEMPERATURE STABLE COMPOSITION OF FAMOTIDINE FOR INTRAVENOUS BOLUS INJECTION AND METHOD THEREOF.” and a PCT Application no. PCT/IB2023/055159 filed on May 19, 2023, and titled “A PREMIXED ROOM TEMPERATURE STABLE COMPOSITION OF FAMOTIDINE FOR INTRAVENOUS BOLUS INJECTION AND METHOD THEREOF.”

### FIELD OF INVENTION

Embodiments of the present disclosure relate to the field of medicinal preparations, and more particularly to a premixed room temperature stable composition of famotidine for intravenous bolus injection and method thereof.

### BACKGROUND

Famotidine is a medicine used to treat patients whose stomach produces excess acid or have ulcers. The famotidine has an ability to inhibit gastric acid secretion through a histamine H2-receptor. The histamine H2-receptor includes blocking agents capable of inhibiting secretion of gastric acid induced by gastrin histamine, methacholine or food. Method of administration of the famotidine may include intravenous bolus and intravenous infusion. The famotidine is a basic compound having an acid dissociation constant (pKa) of about 7.1 and shows potential of hydrogen (pH) dependent solubility and stability characteristics.

The famotidine exhibits a low stability at an acidic pH while the solubility extremely decreases at a neutral pH range where the stability is high. The famotidine undergoes extensive hydrolytic degradation to form mainly famotidine sulfamoyl propenamide, famotidine propenamide and famotidine propionic acid. The famotidine is also susceptible to oxidative degradation to form mainly famotidine disulfide and famotidine sulfoxide. As per the pharmaceutical standards, an acceptance criterion of not more than 5.0% for the sum of the peak areas for famotidine sulfamoyl propanamide, famotidine propenamide and famotidine propionic acid is being followed. Further it is desired to limit the level of oxidative impurities such as famotidine disulfide and famotidine sulfoxide to not more than 1.0%, preferably not more than 0.5% during shelf life.

Currently, famotidine injections available in the market includes a sterile concentrated solution with a concentration of 10 milligrams per milliliter. The sterile concentrated solution has a potential of hydrogen (pH) value of 5.3 to achieve the desired aqueous solubility. The sterile concentrated solution is unstable and therefore needs to be stored under refrigerated conditions to achieve longer shelf life. Refrigeration and storage of the sterile concentrated solution requires special handling. Further, due to hypotonic and sterile concentrated solution nature, of the sterile concentrated solution, the famotidine requires aseptic dilution with a compatible intravenous solution before administration. The sterile concentrated solution may be administered over a period of at least two minutes as intravenous bolus injection.

The famotidine injections available in the market also include a sterile solution with a concentration of 20 milligrams per 50 milliliter. Such a premixed solution is intended for administration as an intravenous infusion over a duration ranging between 15 minutes and 30 minutes, however the solution is unsuitable for intravenous bolus injection due to high volume required to be administered. Currently, the medical field lacks the famotidine injections that are suitable for intravenous bolus administration readily without any dilution, which can be stored at room temperature.

Further, the need for prior dilution of the famotidine injections may have several associated problems. The associated problems may include contamination of the famotidine injections, product wastage, possibility of dosage miscalculation, and a greater chance of needle sticks to medical personnel. The prior dilution may also produce medical wastes in the form of vials, needles and bags.

Hence, there is a need for an improved premixed room temperature stable composition of famotidine for intravenous bolus injection and method thereof to address the aforementioned issue(s).

### BRIEF DESCRIPTION

In accordance with an embodiment of the present disclosure, a premixed room temperature stable composition of famotidine for intravenous bolus injection is provided. The composition includes 0.20 to 0.50 percentage by weight famotidine. The composition also includes a buffering agent. The composition further includes one or more tonicity adjusting agents. The composition includes potential of hydrogen value between 5.7 and 6.4. In accordance with another embodiment of the present disclosure, a method for preparing a premixed room temperature stable composition of famotidine for intravenous bolus injection is provided. The method includes mixing famotidine and a buffering agent to obtain a first solution. The famotidine includes a concentration ranging between 2 milligrams per millilitres to 5 milligrams per millilitres. The method also includes dissolving one or more tonicity adjusting agents to the first solution to obtain a second solution. The second solution includes osmolality ranging between 260 milliosmoles and 330 milliosmoles. The method further includes adjusting potential of hydrogen value of the second solution in between 5.7 and 6.4 by one or more pH adjusting agents thereby preparing the ready to use famotidine injection.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF THE DRAWINGS

The disclosure will be described and explained with additional specificity and detail with the accompanying figures in which:

FIG. 1 is a block diagram representation of a premixed room temperature stable composition of famotidine for intravenous bolus injection in accordance with an embodiment of the present disclosure; and

FIG. 2 is a flow chart representing the steps involved in a method of preparation of premixed room temperature stable composition of famotidine for intravenous bolus injection in accordance with an embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure.

The terms “comprises”, “comprising”, or any other variations thereof, are intended to cover a non-exclusive inclusion, such that a process or method that comprises a list of steps does not include only those steps but may include other steps not expressly listed or inherent to such a process or method. Similarly, one or more devices or sub-systems or elements or structures or components preceded by “comprises . . . a” does not, without more constraints, preclude the existence of other devices, sub-systems, elements, structures, components, additional devices, additional sub-systems, additional elements, additional structures, or additional components. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

In the following specification and the claims, reference will be made to a number of terms, which shall be defined to have the following meanings. The singular forms “a”, “an”, and “the” include plural references unless the context clearly dictates otherwise.

Embodiments of the present disclosure relate to a premixed room temperature stable composition of famotidine for intravenous bolus injection and method thereof. The composition includes 0.20 to 0.50 percentage by weight famotidine. The composition also includes a buffering agent. The composition further includes one or more tonicity adjusting agents. The composition includes potential of hydrogen value between 5.7 and 6.4.

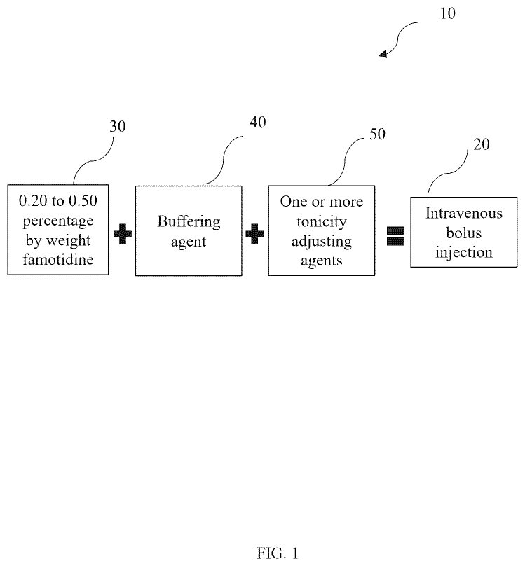

FIG. 1 is a block diagram representation of a premixed room temperature stable composition 10 of famotidine 30 for intravenous bolus injection 20 and method thereof in accordance with an embodiment of the present disclosure. The composition 10 includes 0.20 to 0.50 percentage by weight famotidine 30. The composition 10 also includes a buffering agent 40. The composition 10 further includes one or more tonicity adjusting agents 50. The composition 10 includes potential of hydrogen value between 5.7 and 6.4. In one embodiment, the famotidine 30 may include concentration between 2 milligrams per milliliters and 5 milligrams per milliliters. In such an embodiment, preferable concentration of the famotidine 30 may be 4 milligrams per milliliters.

Further, in one embodiment, the buffering agent 40 may include L-aspartic acid adapted to resist change in potential of hydrogen (pH) value of the composition 10. In one embodiment, the buffering agent 40 may include 0.08 to 0.20 percentage by weight. In one embodiment, the one or more tonicity adjusting agents 50 may include at least one of mannitol, sodium chloride and dextrose to adjust tonicity of the composition 10.

Furthermore, in some embodiment, the composition 10 may include sodium hydroxide to adjust potential of hydrogen (pH) value of the composition 10. In such an embodiment, the sodium hydroxide solution used for the pH adjustment preferably may have a normality of 0.1. In a specific embodiment, the composition 10 may include an antioxidant adapted to minimize formation of oxidative impurities including at least one of famotidine disulphide.

Also, in one embodiment, the antioxidant may include thiol group containing antioxidant chosen from monothioglycerol, L-Cysteine, and the like. In such an embodiment, the monothioglycerol may have percentage by weight between 0.025 to 0.1 with respect to the composition 10. In some embodiments, the composition 10 may include a preservative to prevent microbial contamination of the composition 10. In such an embodiment, the preservative may include at least one of benzyl alcohol, and parabens.

Composition 10 having famotidine concentration of 20 milligrams per 5 millilitres (20 mg/5ml) is shown in table 1.

TABLE 1
Composition 10 having famotidine concentration
of 20 milligrams per 5 millilitres (20 mg/5 ml)

| | | | Formula 1: | |
|---|---|---|---|---|
| | | | 20 mg/5 ml (4 mg/ml) | |
| Sr. | | | Premixed product | |
| No | Ingredient | Function | mg/mL | % w/v |
| 1 | Famotidine 30 USP | Active substance | 4.0 | 0.40 |
| 2 | L-aspartic acid, USP | Buffering agent 40 | 1.6 | 0.16 |
| 3 | Mannitol, USP | Tonicity adjusting | 8.0 | 0.80 |
| 4 | Sodium chloride, USP | agents 50 | 7.0 | 0.70 |
| 5 | Sodium hydroxide, NF | pH adjusting agent | q.s. to adjust pH | |
| | | | to about 5.8-6.2 | |
| 6 | Water for Injection, | Vehicle | q.s. to | q.s. to |
| | USP | | 1 mL | 100.00% |

| | | |
|---|---|---|
| 7 | Nitrogen, NF | Processing aid |

The processing aid, nitrogen may be used to reduce dissolved oxygen levels in the composition 10, and to reduce oxygen content in vial headspace and also for aiding filtration process by pressurization of bulk solution. Stability of the composition 10 having famotidine concentration of 4 milligrams per milliliters at different pH ranging pH 5.8 to pH 6.4 in accelerated (40° Celcius (C)/75% Relative Humidity (RH)) and long-term stability (25° C./60% RH) conditions at inverted orientation of the vial is provided in Table 2, Table 3 and Table 4.

Stability data of the composition 10 having famotidine concentration of 4 milligrams per millilitres at a pH of about 5.8 is shown in Table 2.

TABLE 2
Stability data of the composition 10 having famotidine concentration
of 4 milligrams per millilitres at a pH of 5.8

| | | Accelerated stability condition | | | Long term stability condition | | |
|---|---|---|---|---|---|---|---|
| | | 40° C./75% RH | | | 25° C./60% RH | | |
| Test | Initial | 1 Month | 2 Month | 3 Month | 1 Month | 2 Month | 3 Month |
| Description | Clear | Clear | Clear | Clear | Clear | Clear | Clear |
| | Colourless | Colourless | Colourless | Colourless | Colourless | Colourless | Colourless |
| | solution | solution | solution | solution | solution | solution | solution |
| pH | 5.89 | 6.22 | 6.09 | 6.08 | 6.05 | 5.99 | 5.98 |
| Osmolality | 282 | 291 | 289 | 286 | 290 | 288 | 288 |
| (mosmoles/kg) | | | | | | | |
| Assay | 97.7 | 100.1 | 98.4 | 96.3 | 100.8 | 101.5 | 100.0 |
| (% w/v) | | | | | | | |

Related substances (% w/w)

| | | | | | | | |
|---|---|---|---|---|---|---|---|
| Sulfoxide | 0.01 | 0.01 | BDL | 0.03 | 0.02 | 0.01 | 0.01 |
| Impurity | | | | | | | |
| Famotidine | BDL | 0.02 | 0.06 | 0.19 | BDL | BDL | 0.02 |
| Amidine | | | | | | | |
| Propanamide | BDL | 0.2 | 0.63 | 1.16 | 0.02 | 0.03 | 0.05 |
| impurity | | | | | | | |
| USP Impurity-D | | | | | | | |
| Sulfamoyl | 0.08 | 0.81 | 1.47 | 1.60 | 0.25 | 0.36 | 0.47 |
| propenamide | | | | | | | |
| USP Impurity-C | | | | | | | |
| Propionic acid | 0.04 | 0.02 | BDL | BDL | BDL | BDL | BDL |
| impurity | | | | | | | |
| USP Impurity-F | | | | | | | |
| Impurity-G | 0.08 | 0.06 | 0.06 | 0.03 | 0.06 | 0.07 | 0.04 |
| Famotidine | 0.06 | 0.17 | 0.22 | 0.41 | 0.10 | 0.08 | 0.11 |
| disulphide | | | | | | | |
| USP Impurity-E | | | | | | | |
| Impurity at RRT | BDL | 0.03 | 0.12 | 0.24 | BDL | 0.02 | 0.12 |
| about 0.85 | | | | | | | |
| Impurity at RRT | 0.02 | 0.06 | 0.08 | 0.11 | 0.03 | 0.03 | 0.06 |
| 2.30 | | | | | | | |
| Any unspecified | 0.10 | 0.03 | 0.05 | BDL | BDL | 0.07 | BDL |
| impurity | | | | | | | |
| Total Impurities | 0.31 | 1.34 | 2.79 | 4.08 | 0.47 | 0.57 | 0.86 |

Stability data of the composition 10 having famotidine concentration of 4 milligrams per milliliters at a pH of about 6.2 is shown in Table 3.

TABLE 3
Stability data of the composition 10 having famotidine concentration
of 4 milligrams per milliliters at a pH of 6.2

| | | Accelerated stability condition | | | Long term stability condition | | |
|---|---|---|---|---|---|---|---|
| | | 40° C./75% RH | | | 25° C./60% RH | | |
| Test | Initial | 1 Month | 2 Month | 3 Month | 1 Month | 2 Month | 3 Month |
| Description | Clear | Clear | Clear | Clear | Clear | Clear | Clear |
| | Colourless | Colourless | Colourless | Colourless | Colourless | Colourless | Colourless |
| | solution | solution | solution | solution | solution | solution | solution |
| pH | 6.21 | 6.35 | 6.42 | 6.36 | 6.29 | 6.34 | 6.37 |
| Osmolality | 286 | 293 | 294 | 291 | 293 | 289 | 291 |
| (mosmoles/kg) | | | | | | | |
| Assay (% w/v) | 98.7 | 99.6 | 99.2 | 96.7 | 100.8 | 101.0 | 100.2 |

Related substances (% w/w)

| | | | | | | | |
|---|---|---|---|---|---|---|---|
| Sulfoxide | 0.01 | 0.03 | BDL | 0.03 | 0.01 | BDL | 0.02 |
| Impurity | | | | | | | |
| Famotidine | BDL | 0.03 | 0.08 | 0.23 | BDL | BDL | 0.02 |
| Amidine | | | | | | | |
| Propanamide | BDL | 0.10 | 0.31 | 0.54 | 0.01 | 0.04 | 0.05 |
| impurity | | | | | | | |
| USP Impurity-D | | | | | | | |
| Sulfamoyl | 0.06 | 0.54 | 1.05 | 1.21 | 0.18 | 0.29 | 0.36 |
| propenamide | | | | | | | |
| USP Impurity-C | | | | | | | |
| Propionic acid | 0.03 | 0.04 | BDL | BDL | BDL | BDL | BDL |
| impurity | | | | | | | |
| USP Impurity-F | | | | | | | |
| Impurity-G | 0.08 | 0.05 | 0.06 | 0.03 | 0.05 | 0.07 | 0.04 |
| USP Impurity-E | 0.06 | 0.42 | 0.38 | 0.70 | 0.11 | 0.15 | 0.17 |
| Famotidine | | | | | | | |
| disulfide | | | | | | | |
| Impurity at RRT | BDL | 0.03 | 0.12 | 0.24 | BDL | 0.02 | 0.12 |
| about 0.85 | | | | | | | |
| Impurity at RRT | 0.02 | 0.08 | 0.13 | 0.16 | 0.04 | 0.04 | 0.09 |
| 2.30 | | | | | | | |
| Any unspecified | 0.10 | 0.03 | 0.05 | BDL | BDL | 0.07 | BDL |
| impurity | | | | | | | |
| Total Impurities | 0.30 | 1.48 | 2.39 | 3.46 | 0.39 | 0.65 | 0.91 |

Stability data of the composition 10 having famotidine concentration of 4 milligram per milliliter at a pH of 6.4 is shown in table 4.

TABLE 4
Stability data of the composition 10 having famotidine concentration
of 4 milligram per milli liter at a pH of 6.4

| | | Accelerated stability condition | | | Long term stability condition | | |
|---|---|---|---|---|---|---|---|
| | | 40° C./75% RH | | | 25° C./60% RH | | |
| Test | Initial | 1 Month | 2 Month | 3 Month | 1 Month | 2 Month | 3 Month |
| Description | Clear | Clear | Clear | Clear | Clear | Clear | Clear |
| | Colourless | Colourless | Colourless | Colourless | Colourless | Colourless | Colourless |
| | solution | solution | solution | solution | solution | solution | solution |
| PH | 6.38 | 6.40 | 6.53 | 6.41 | 6.41 | 6.47 | 6.44 |
| Osmolality | 279 | 290 | 285 | 289 | 287 | 285 | 286 |
| (mosmoles/kg) | | | | | | | |
| Assay (% w/v) | 97.4 | 98.1 | 97.3 | 94.8 | 97.5 | 93.9 | 97.1 |

Related substances (% w/w)

| | | | | | | | |
|---|---|---|---|---|---|---|---|
| Sulfoxide | 0.01 | 0.01 | BDL | 0.04 | 0.01 | 0.02 | 0.03 |
| Impurity | | | | | | | |
| Famotidine | BDL | 0.03 | 0.08 | 0.24 | BDL | BDL | 0.03 |
| Amidine | | | | | | | |
| Propanamide | BDL | 0.09 | 0.25 | 0.49 | 0.02 | 0.04 | 0.04 |
| impurity | | | | | | | |
| USP Impurity-D | | | | | | | |
| Sulfamoyl | 0.05 | 0.48 | 0.92 | 1.13 | 0.15 | 0.24 | 0.31 |
| propenamide | | | | | | | |
| USP Impurity-C | | | | | | | |
| Propionic acid | 0.04 | 0.04 | BDL | BDL | BDL | BDL | BDL |
| impurity | | | | | | | |
| USP Impurity-F | | | | | | | |
| Impurity-G | 0.07 | 0.05 | 0.06 | 0.03 | 0.05 | 0.06 | 0.04 |
| USP Impurity-E | 0.06 | 0.24 | 0.44 | 0.94 | 0.11 | 0.17 | 0.24 |
| Famotidine | | | | | | | |
| disulphide | | | | | | | |
| Impurity at RRT | BDL | 0.03 | 0.11 | 0.23 | BDL | 0.03 | 0.11 |
| about 0.85 | | | | | | | |
| Impurity at RRT | 0.02 | 0.09 | 0.14 | 0.15 | 0.04 | 0.05 | 0.10 |
| 2.30 | | | | | | | |
| Any unspecified | 0.10 | 0.03 | 0.05 | BDL | BDL | 0.07 | BDL |
| impurity | | | | | | | |
| Total Impurities | 0.28 | 0.95 | 2.29 | 3.71 | 0.36 | 0.72 | 0.99 |

Based on the table 2, table 3 and table 4, it may be observed that the famotidine 30 remained in the composition 10 throughout the assessed stability period between the pH 5.8 to pH 6.4 without any precipitation. Though levels of hydrolytic impurities such as sulfamoyl propenamide and propanamide were observed slightly higher at pH 5.8 compared to pH 6.2 and pH 6.4, the levels were within acceptable limits. The level of famotidine disulphide was observed at a higher level in pH 6.4 compared to pH of 6.2 and 5.8.

Composition 10 having famotidine concentration of 4 milligram per millilitre with an antioxidant, monothioglycerol at a concentration of 0.1 and 1 milligram per milliliter is shown in table 5.

TABLE 5
Composition 10 having famotidine concentration of 4 milligram per millilitre with
monothioglycerol having concentration of 0.1 or 1 milligram per milliliter

| | | | Formula 2: | | Formula 3: | |
|---|---|---|---|---|---|---|
| | | | Premixed product | | Premixed product | |
| | | | with | | with | |
| | | | 0.05 mg/mL | | 1 mg/mL | |
| | | | antioxidant | | antioxidant | |
| Sr. No. | Ingredient | Function | mg/mL | % w/v | mg/mL | % w/v |
| 1 | Famotidine 30 USP | Active | 4.0 | 0.40 | 4.0 | 0.40 |
| | | substance | | | | |
| 2 | L-aspartic acid, | Buffering agent | 1.6 | 0.16 | 1.6 | 0.16 |
| | USP | 40 | | | | |
| 3 | Mannitol, USP | Tonicity | 8.0 | 0.80 | 8.0 | 0.80 |
| | Sodium chloride, | adjusting | 7.0 | 0.70 | 7.0 | 0.70 |
| 4 | USP | agents 50 | | | | |
| 5 | Monothioglycerol, | Antioxidant | 0.05 | 0.005 | 1.0 | 0.1 |
| | NF | | | | | |
| 6 | Sodium hydroxide, | pH adjusting | q.s. to adjust pH to | | q.s. to adjust pH to | |
| | NF | agent | about 6.2 | | about 6.2 | |
| | | | (Range 5.7 to 6.4) | | (Range 5.7 to 6.4) | |
| 7 | Water for | Vehicle | q.s. | q.s. | q.s. | q.s. |
| | Injection, USP | | to 1 mL | to 100.0% | to 1 mL | to 100.0% |

| | | | | |
|---|---|---|---|---|
| 8 | Nitrogen, NF | Processing aid | Processing aid | Processing aid |

Stability data of the composition 10 having famotidine concentration of 4 milli grams per millilitre along with monothioglycerol concentration of 1 milligrams per millilitre is shown in table 6.

TABLE 6
Stability data of the composition 10 having famotidine concentration of 4 milligram
per milliliter with monothioglycerol at 1 milligram per milliliter.

| | | 40° C./75% RH | | | 25° C./60% RH | |
|---|---|---|---|---|---|---|
| Test | Initial | Week 2 | 1 Month | 2 Month | 1 Month | 2 Month |
| Description | Clear | Clear | Clear | Clear | Clear | Clear |
| | Colourless | Colourless | Colourless | Colourless | Colourless | Colourless |
| | solution | solution | solution | solution | solution | solution |
| pH | 6.26 | 6.26 | 6.22 | 6.32 | 6.23 | 6.26 |
| Osmolality | 299 | 297 | 295 | 299 | 297 | 304 |
| (mOsm/kg) | | | | | | |
| Assay (% w/v) | 100.9 | 99.7 | 98.3 | 94.8 | 99.8 | 99.0 |

Related substances (% w/w)

| | | | | | | |
|---|---|---|---|---|---|---|
| Sulfoxide | BDL | BDL | 0.08 | 0.09 | 0.18 | 0.12 |
| Impurity | | | | | | |
| Famotidine | BDL | BDL | 0.02 | BDL | 0.03 | BDL |
| Amidine | | | | | | |
| Propanamide | 0.01 | 0.08 | 0.20 | 0.38 | 0.04 | 0.05 |
| impurity | | | | | | |
| USP Impurity-D | | | | | | |
| Sulfamoyl | 0.09 | 0.33 | 0.72 | 1.00 | 0.24 | 0.30 |
| propanamide | | | | | | |
| USP Impurity-C | | | | | | |
| Propionic acid | 0.03 | BDL | BDL | 0.09 | BDL | 0.03 |
| impurity | | | | | | |
| USP Impurity-F | | | | | | |
| USP Impurity-G | BDL | 0.06 | 0.06 | 0.03 | 0.08 | 0.04 |
| Famotidine | 0.03 | BDL | BDL | BDL | BDL | BDL |
| disulphide | | | | | | |
| USP Impurity-E | | | | | | |
| Impurity at RRT | 0.021 | 0.04 | 0.07 | 0.12 | 0.02 | 0.04 |
| about 0.85 | | | | | | |
| Impurity at RRT | 0.08 | 0.25 | 0.06 | 0.33 | 0.10 | 0.13 |
| about 1.80 | | | | | | |
| Impurity at RRT | 0.07 | 0.08 | 0.14 | 0.39 | 0.08 | 0.11 |
| about 2.30 | | | | | | |
| Any unspecified | ND | 0.07 | 0.11 | 0.12 | 0.06 | 0.07 |
| impurity | | | | | | |
| Total impurities | 0.29 | 0.94 | 1.5 | 2.50 | 0.9 | 0.83 |

From the table 6, it has been observed that inclusion of the monothioglycerol, minimised the presence of oxidative degradant, famotidine disulphide in the composition 10. Even though the impurity levels at RRT about 1.80 and RRT about 2.30 is found increasing in accelerated stability condition, the impurity levels at room temperature are found to be satisfactory.

In an exemplary embodiment, multi-dose vial composition 10 of multi-dose vial having a preservative and famotidine concentration of 4 milligrams per millilitres is shown in table 7.

TABLE 7
Composition 10 of multidose vial having a preservative and
famotidine concentration of 4 milligrams per millilitres

| | | | Formula 4: | |
|---|---|---|---|---|
| | | | Premixed, multidose | |
| | | | vial having a | |
| Sr. | | | preservative | |
| No. | Ingredient | Function | mg/mL | % w/v |
| 1 | Famotidine (30) USP | Active substance | 4.0 | 0.4 |
| 2 | L-aspartic acid, USP | Buffering agent 40 | 1.6 | 0.16 |
| 3 | Mannitol, USP | Tonicity adjusting | 8.0 | 0.8 |
| 4 | Sodium chloride, USP | agents 50 | 7.0 | 0.7 |
| 5 | Benzyl alcohol NF | Preservative | 3.6 to | 0.36% to |
| | | | 9.0 | 0.90% |
| 6 | Sodium hydroxide, NF | pH adjusting | q.s. to adjust pH | |
| | | agent | to about 6.2 | |
| | | | (Range 5.7 to 6.4) | |
| 7 | Water for Injection, | Vehicle | q.s. to | q.s. to |
| | USP | | 1 mL | 100.00% |

| | | | |
|---|---|---|---|
| 8 | Nitrogen, NF | Processing aid | Processing aid |

Stability data of the composition 10 of 10 mL fill multi-dose vials of Formula 3 having famotidine concentration of 4 mg/mL and benzyl alcohol at 9 mg/mL is shown in table 8.

TABLE 8
Stability data of the composition 10 of multi-dose vials having
famotidine concentration of 4 mg/mL and 9 mg/mL benzyl alcohol

| | | 40° C./75% RH | | 25° C./60% RH | |
|---|---|---|---|---|---|
| Test | Initial | 1 Month | 3 Month | 1 Month | 3 Month |
| Description | Clear | Clear | Clear | Clear | Clear |
| | Colourless | Colourless | Colourless | Colourless | Colourless |
| | solution | solution | solution | solution | solution |
| pH | 6.18 | 6.17 | 6.20 | 6.16 | 6.19 |
| Osmolality (mOsm/kg) | 274 | 274 | 282 | 274 | 283 |
| Assay (% w/v) | 99.7 | 97.8 | 95.0 | 98.6 | 98.8 |
| Benzyl Alcohol | 99.2 | 98.2 | 98.1 | 98.0 | 99.5 |
| (% label claim) | | | | | |
| Benzaldehyde Content | 0.01 | 0.07 | 0.02 | 0.03 | 0.01 |

Related substances (% w/w)

| | | | | | |
|---|---|---|---|---|---|
| Sulfoxide Impurity | 0.05 | 0.09 | 0.18 | 0.06 | 0.07 |
| Famotidine Amidine | BDL | 0.02 | 0.07 | BDL | BDL |
| Propanamide impurity | BDL | 0.12 | 0.65 | 0.02 | 0.05 |
| USP Impurity-D | | | | | |
| Sulfamoyl propanamide | 0.07 | 0.53 | 1.25 | 0.19 | 0.38 |
| USP Impurity-C | | | | | |
| Propionic acid impurity | 0.03 | 0.02 | BDL | 0.01 | BDL |
| USP Impurity-F | | | | | |
| USP Impurity-G | 0.08 | 0.06 | 0.04 | 0.04 | 0.04 |
| Famotidine disulphide | 0.03 | 0.26 | 0.71 | 0.04 | 0.21 |
| USP Impurity-E | | | | | |
| Impurity at RRT about | 0.09 | 0.04 | 0.25 | 0.01 | 0.13 |
| 0.85 | | | | | |
| Impurity at RRT about | BDL | 0.07 | 0.17 | 0.04 | BDL |
| 1.80 | | | | | |
| Impurity at RRT about | 0.01 | 0.04 | 0.09 | 0.02 | BDL |
| 2.30 | | | | | |
| Any unspecified impurity | ND | ND | 0.12 | ND | 0.06 |
| Total impurities | 0.34 | 1.15 | 4.18 | 0.32 | 1.08 |

Composition 10 having famotidine concentration of 2 milligrams per millilitres and 5 milligrams per millilitres is shown in table 9.

TABLE 9
Composition 10 having famotidine concentration of 2 milligrams per millilitres
and famotidine concentration of 5 milligrams per millilitres.

| | | | Formula 5: | | Formula 6: | |
|---|---|---|---|---|---|---|
| | | | Premixed product | | Premixed product | |
| | | | having | | having | |
| | | | 2 mg/mL | | 5 mg/mL | |
| | | | Famotidine | | Famotidine | |
| Sr. No. | Ingredient | Function | mg/mL | % w/v | mg/mL | % w/v |
| 1 | Famotidine 30 | Active | 2.0 | 0.2 | 5.0 | 0.5 |
| | USP | substance | | | | |
| 2 | L-aspartic acid, | Buffering | 0.8 | 0.08 | 2.0 | 0.2 |
| | USP | agent 40 | | | | |
| 3 | Mannitol, USP | Tonicity | 4.0 | 0.4 | 50.0 | 5.0 |
| | Sodium chloride, | adjusting | 8.0 | 0.8 | Not used | |
| 4 | USP | agents 50 | | | | |
| 5 | Sodium | pH adjusting | q.s. to adjust pH to | | q.s. to adjust pH to | |
| | hydroxide, NF | agent | about 6.2 | | about 6.2 | |
| | | | (Range 5.7 to 6.4) | | (Range 5.7 to 6.4) | |
| 6 | Water for | Vehicle | q.s. | q.s. | q.s. | q.s. |
| | Injection, USP | | to 1 mL | to 100.0% | to 1 mL | to 100.0% |

| | | | |
|---|---|---|---|
| 7 | Nitrogen, NF | Processing aid | Processing aid |

Stability data of the composition 10 having famotidine concentration of 2 milligrams per millilitres is shown in table 10.

TABLE 10
Stability data of the composition 10 having famotidine
concentration of 2 milligrams per millilitres

| | | 40° C./75% RH | | 25° C./60% RH | |
|---|---|---|---|---|---|
| Parameters | Initial | 1 Month | 3 Month | 1 Month | 3 Month |
| Description | Clear | Clear | Clear | Clear | Clear |
| | colourless | colourless | colourless | colourless | colourless |
| | solution, free | solution, free | solution, free | solution, free | solution, free |
| | from visible | from visible | from visible | from visible | from visible |
| | particles | particles | particles | particles | particles |
| pH | 6.00 | 6.05 | 6.21 | 6.05 | 6.10 |
| Osmolality | 243 | 248 | 251 | 244 | 246 |
| (mOsm/kg) | | | | | |

Related substances (% w/w)

| | | | | | |
|---|---|---|---|---|---|
| Famotidine Amidine | BDL | BDL | BDL | BDL | BDL |
| USP Impurity-D | BDL | 0.08 | 0.53 | BDL | BDL |
| Propanamide | | | | | |
| impurity | | | | | |
| USP Impurity-C | 0.04 | 0.62 | 1.67 | 0.16 | 0.38 |
| Sulfamoyl | | | | | |
| Propanamide | | | | | |
| USP Impurity-F | BDL | BDL | 0.22 | BDL | BDL |
| Propionic acid | | | | | |
| impurity | | | | | |
| Total Impurities | 0.04 | 1.11 | 2.65 | 0.22 | 0.38 |

Stability data of the composition 10 having famotidine concentration of 5 milligrams per millilitres is shown in table 11.

TABLE 11
Stability data of the composition 10 having famotidine
concentration of 5 milligrams per millilitres

| | Product pH 6.0 | | | |
|---|---|---|---|---|
| Stability test | | 25° C./60% RH | | |
| parameters | Initial | 1 Month | 2 Month | 6 Month |
| Description | Clear | Clear | Clear | Clear |
| | colourless | colourless | colourless | colourless |
| | solution, | solution, | solution, | solution, |
| | free from | free from | free from | free from |
| | visible | visible | visible | visible |
| | particles | particles | particles | particles |
| pH | 6.00 | 6.04 | 6.05 | 6.09 |

Related substances (% w/w)

| | | | | |
|---|---|---|---|---|
| Famotidine Amidine | BDL | BDL | BDL | BDL |
| USP Impurity-D | BDL | BDL | BDL | 0.08 |
| Propanamide impurity | | | | |
| USP Impurity-C | 0.05 | 0.18 | 0.27 | 0.76 |
| Sulfamoyl propanamide | | | | |
| USP Impurity-F | BDL | BDL | BDL | 0.08 |
| Propionic acid impurity | | | | |
| Highest unspecified | BDL | BDL | BDL | 0.11 |
| Impurity | | | | (RRT- |
| | | | | 0.825) |
| Total Impurities | 0.05 | 0.18 | 0.27 | 1.1 |

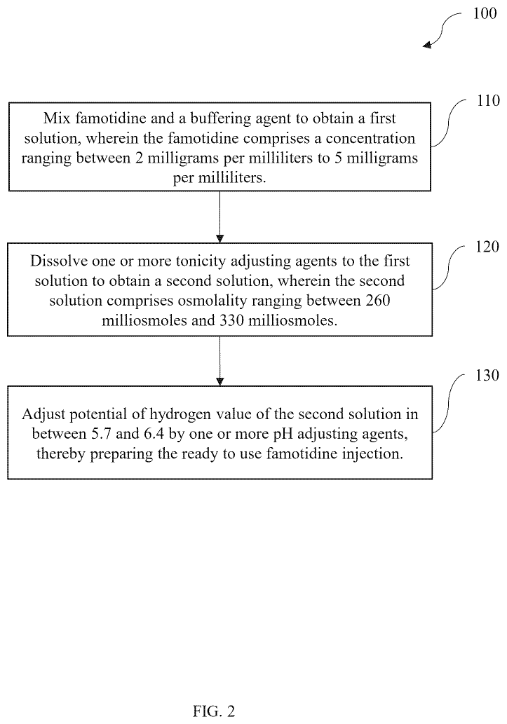

FIG. 2 is a flow chart representing the steps involved in a method 100 of preparation of a premixed room temperature stable composition of famotidine for intravenous bolus injection in accordance with an embodiment of the present disclosure. The method 100 includes mixing famotidine and a buffering agent to obtain a first solution in step 110. The famotidine includes a concentration ranging between 2 milligrams per milliliters to 5 milligrams per milliliters. The method 100 also includes dissolving one or more tonicity adjusting agents to the first solution to obtain a second solution in step 120. The second solution includes osmolality ranging between 260 milliosmoles and 330 milliosmoles. The method 100 further includes adjusting potential of hydrogen value of the second solution in between 5.7 and 6.4 by adding one or more pH adjusting agents, thereby preparing the ready to use famotidine injection 130.

Further, in one embodiment, the second solution may be made into batch volume. Aseptic filling of the second solution made into the batch volume may be done after filtering by sterilizing grade filters. In one embodiment, the one or more pH adjusting agents may include at least one of a sodium hydroxide solution and an aspartic acid solution. In such an embodiment, the sodium hydroxide solution may be having a normality of 0.1 N and the aspartic acid solution may be having a concentration of 0.5%. In one embodiment, the second solution may be cooled down to a temperature ranging between 20 degree celsius and 30 degree celsius after adjusting the potential of hydrogen value.

Exemplary steps involved in preparation of the ready to use famotidine injection having concentration of 20 milligrams per 5 milli liters is given below. Collect water for injection equivalent to 110% of the batch size and sparge with nitrogen gas to reduce the dissolved oxygen level to <1 ppm. Collect water for injection from the previous step equivalent to 60% of the batch size and heat to approximately 60° C.-70° C. Add and dissolve L-Aspartic acid, USP by mixing for Approx. 20 minutes. Cool the solution temperature to 20° C.-30° C. Maintain Nitrogen gas sparging of the solution throughout the process except during material addition. Add and dissolve Famotidine by mixing for Approx. 40 minutes to obtain a clear solution. Add and dissolve the tonicity adjusting agent, Mannitol and/or sodium chloride one after other by mixing for approx. 10 minutes after each ingredient addition. Adjust the pH to about 6.2 using 0.1N sodium hydroxide solution and/or 0.5% Aspartic acid solution and then make up the volume to batch volume. Filter the solution using suitable 0.2 micron sterilizing grade filters and fill 5 mL of the filtered solution in 5 mL, 13 mm neck, USP type 1 Glass vial. Blanket the vial headspace using Nitrogen gas to minimize the headspace oxygen level, preferably to less than 10%. Stopper the vials using 13 mm butyl rubber stopper.

Various embodiments of the composition of a premixed room temperature stable composition of famotidine for intravenous bolus injection and method thereof described above enable various advantages. The composition is suitable for intravenous bolus administration without any prior dilution. The composition is capable of being stored in room temperature, thereby eliminating the need of refrigeration. Also, the composition is suitable for intravenous bolus administration. Eliminating the need of prior dilution negates the chances of contamination of the composition during the dilution, product wastage, dosage miscalculations, needle sticks to the medical personnel and production of medical wastes in the form of vials, needles and bags. Further, room temperature stable injection gives an opportunity to store and transport the ready to use famotidine injection more efficiently.

It will be understood by those skilled in the art that the foregoing general description and the following detailed description are exemplary and explanatory of the disclosure and are not intended to be restrictive thereof. While specific language has been used to describe the disclosure, any limitations arising on account of the same are not intended.

The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, the order of processes described herein may be changed and are not limited to the manner described herein. Moreover, the actions of any flow diagram need not be implemented in the order shown; nor do all the acts need to be necessarily performed. Also, those acts that are not dependent on other acts may be performed in parallel with the other acts. The scope of embodiments is by no means limited by these specific examples.

We claim:

1. A premixed room temperature stable composition of famotidine for intravenous bolus injection comprising:
0.20 to 0.50 percentage by weight famotidine;
a buffering agent; and
one or more tonicity adjusting agents,
wherein the composition comprises potential of hydrogen value between 5.7 and 6.4.

2. The composition as claimed in claim 1, wherein the famotidine comprises concentration between 2 milli grams per milli liters and 5 milli grams per milli liters.

3. The composition as claimed in claim 1, wherein the buffering agent comprises 0.08 to 0.20 percentage by weight, wherein the buffering agent comprises L-aspartic acid adapted to resist change in potential of hydrogen (pH) value of the composition.

4. The composition as claimed in claim 1, wherein the one or more tonicity adjusting agents comprises at least one of mannitol, sodium chloride and dextrose to adjust tonicity of the composition between 260 milliosmoles per kilograms and 330 milliosmoles per kilograms

5. The composition as claimed in claim 1, comprising sodium hydroxide to adjust potential of hydrogen (pH) value of the composition, wherein the sodium hydroxide comprises a normality of 0.1.

6. The composition as claimed in claim 1, comprising an antioxidant to minimize formation of oxidative impurities comprising famotidine disulphide impurity, wherein the antioxidant comprises monothioglycerol comprising percentage by weight between 0.025 to 0.1 with respect to the composition.

7. The composition as claimed in claim 1, comprising a preservative to prevent microbial contamination of the composition, wherein the preservative comprises at least one of benzyl alcohol, and parabens.

8. A method of preparation of a premixed room temperature stable composition of famotidine for intravenous bolus injection comprising:
mixing famotidine and a buffering agent to obtain a first solution, wherein the famotidine comprises a concentration ranging between 2 milligrams per milliliters to 5 milligrams per milliliters;
dissolving one or more tonicity adjusting agents to the first solution to obtain a second solution, wherein the second solution comprises osmolality ranging between 260 milliosmoles and 330 milliosmoles; and
adjusting potential of hydrogen value of the second solution in between 5.7 and 6.4 by one or more pH adjusting agents), thereby preparing the ready to use famotidine injection.

9. The method as claimed in claim 7, wherein the one or more pH adjusting agents comprises at least one of a sodium hydroxide solution and an aspartic acid solution.
